(19) United States
(12) Patent Application Publication
(10) Pub. No.: US 2009/0069689 A1
(43) Pub. Date: Mar. 12, 2009

(54) ULTRASONIC PROBE AND ULTRASONIC IMAGING APPARATUS

(76) Inventor: Hiroshi Isono, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/205,358

(22) Filed: Sep. 5, 2008

(30) Foreign Application Priority Data
Sep. 6, 2007 (JP) 2007-231709

(51) Int. Cl. A61B 8/14

(52) U.S. Cl. 600/459

(57) ABSTRACT

An ultrasonic probe includes: a composite piezoelectric element including piezoelectric elements arranged through gaps on a plane and a filler filled in each of the gaps; an adhesive layer positioned in an ultrasonic wave irradiating direction orthogonal to the plane, the adhesive layer being in contact with one plate surface of the composite piezoelectric element; and a complete reflector layer joined to the composite piezoelectric element through the adhesive layer to reflect an ultrasonic elastic oscillation generated in the composite piezoelectric element substantially toward the composite piezoelectric element plate surface. The composite piezoelectric element has a plate-like connector portion constituted by only the piezoelectric elements, the plate-like connector portion covering the whole of the composite piezoelectric element.

### CROSS REFERENCE TO RELATED APPLICATIONS

This application claims the benefit of Japanese Patent Application No. 2007-231709 filed Sep. 6, 2007, which is hereby incorporated by reference in its entirety.

### BACKGROUND OF THE INVENTION

The subject matter disclosed herein relates to an ultrasonic probe for exciting an elastic oscillation in a composite piezoelectric clement, the elastic oscillation corresponding to a quarter wavelength of a generated ultrasonic wave, as well as an ultrasonic imaging apparatus.

In ultrasonic imaging apparatuses, it has been constantly tried so that the image quality of tomographic image information is improved. As a method for improving the image quality there sometimes is adopted a method wherein an ultrasonic probe is rendered high in sensitivity and wide in frequency band.

According to a recent ultrasonic probe, an elastic oscillation with the thickness of a piezoelectric element of the ultrasonic probe corresponding to a quarter wavelength is excited in the piezoelectric element and an ultrasonic wave induced by the elastic oscillation is radiated to a subject. The piezoelectric element makes approximately zero the energy of an ultrasonic wave radiated in the direction opposite to the direction where the subject is positioned, increases the energy of the ultrasonic wave radiated to the surface on which the subject is present, and thus attains high sensitivity. For making the piezoelectric element perform a quarter-wave resonance, a complete reflector layer (also called a de-matching layer) of a high acoustic impedance is formed on the plate surface of the piezoelectric element opposed to the piezoelectric element's plate surface on the side where the subject is positioned (see, for example, U.S. Pat. No. 6,685,647).

As the material of the piezoelectric element that performs such a quarter-wave resonance there is used a composite piezoelectric element. The composite piezoelectric element includes piezoelectric elements such as PZT (piezoelectric zirconate titanate) arranged in an array form, with resin being filled in gap portions of the thus-arranged piezoelectric elements. The composite piezoelectric element is approximately equal in electromechanical coupling coefficient to PZT, while its acoustic impedance is small in comparison with that of PZT. By using the composite piezoelectric element as the material of the piezoelectric element that performs a quarter-wave resonance there are attained improvement of sensitivity and a wide band of resonance frequency characteristic.

It is not easy to let a stable and satisfactory quarter-wave resonance be performed with use of the composite piezoelectric element. More particularly, for allowing the composite piezoelectric element to perform a satisfactory quarter-wave resonance stably, it is necessary that the thickness of the adhesive layer present at the boundary between the composite piezoelectric element and the complete reflector layer be made small and uniform.

The thickness of the adhesive layer exerts a great influence on resonance characteristic at the time of performing the quarter-wave oscillation. An example is shown in FIG. 7. FIG. 7 is an explanatory diagram showing the results of simulation performed with respect to frequency bands of a transfer function which an ultrasonic probe possesses, the ultrasonic probe including an acoustic lens, an acoustic matching layer, piezoelectric elements and a complete reflector layer. In FIG. 7, frequency is plotted along the abscissa axis, while the magnitude of frequency response is plotted along the ordinate axis in terms of decibel (dB). A peak value of frequency response is set at 0 dB. This ultrasonic probe has a frequency band with a center frequency in the vicinity of 3 MHz.

Curved lines 81 to 83 shown in FIG. 7 represent frequency bands at adhesive layer thicknesses of 1 μm, 2 μm, and 3 μm, respectively. The frequency bands indicated by the curved lines 81 to 83 become narrower as the adhesive layer thickness changes from 1 μm to 3 μm. For example, as to each of the bandwidths of the curved lines 81 to 83 at a position lower by 6 dB from the peak value, a specific bandwidth obtained by dividing the bandwidth by a center frequency decreases to 80%˜71%. The ultrasonic probe, when having a wide frequency band, can generate a pulse of a short time superior in ring-down characteristic of an ultrasonic waveform, and hence it is possible to improve the resolution of a tomographic image.

Thus, for making the composite piezoelectric element perform a satisfactory quarter-wave resonance stably, it is considered necessary that the thickness of the adhesive layer between the composite piezoelectric element and the complete reflector layer be thin and uniform. In this connection, as shown in the above simulation results, in order to control the adhesive layer thickness on the order of several μm, the opposed adhesive surfaces of the composite piezoelectric element and the complete reflector layer are both specularly polished and smoothed at the time of forming the adhesive layer.

However, according to the above background technique, it is difficult to specularly polish the adhesive surface of the composite piezoelectric element uniformly. More particularly, the adhesive surface of the composite piezoelectric element contains a ceramic material as the material of piezoelectric elements and resin as the filler, and thus two materials different in hardness coexist. It is difficult to specularly polish such an adhesive surface uniformly and diminish concaves and convexes to the order of several μm.

For example, in specularly polishing the adhesive surface of the composite piezoelectric element, the piezoelectric element portion is specularly polished deeply, while the resin portion is specularly polished shallowly. As a result, there occur concaves and convexes on the adhesive surface of the composite piezoelectric element, that is, the adhesive surface becomes no longer flat. Also as to the adhesive layer between the composite electric element and the complete reflector layer, it is thin and difficult to be made thick uniformly because concaves and convexes are present on the adhesive layer.

Thus, it is important how to implement an ultrasonic probe having a composite piezoelectric element whose plate surface opposed to the complete reflector layer can be specularly finished uniformly, as well as an ultrasonic imaging apparatus using such an ultrasonic probe.

### BRIEF DESCRIPTION OF THE INVENTION

It is desirable that the problems described previously are solved.

In a first aspect of the invention there is provided an ultrasonic probe including a composite piezoelectric element, the composite piezoelectric element including piezoelectric elements arranged through gaps on a plane and a filler filled in each of the gaps, an adhesive layer positioned in an ultrasonic wave irradiating direction orthogonal to the plane, the adhesive layer being in contact with one plate surface of the composite piezoelectric element, and a complete reflector layer joined to the composite piezoelectric element through the adhesive layer to reflect an ultrasonic elastic oscillation generated in the composite piezoelectric element substantially toward the composite piezoelectric element plate surface, wherein the composite piezoelectric element has a plate-like connector portion constituted by only the piezoelectric elements, the plate-like connector portion covering the whole of the composite piezoelectric element.

In the invention according to the first aspect, the whole of the composite piezoelectric element plate surface that is in contact with the adhesive layer is covered with the plate-like connector portion constituted by only the piezoelectric elements.

In a second aspect of the invention there is provided, in combination with the above first aspect, an ultrasonic probe wherein the piezoelectric elements in the composite piezoelectric element are arranged in only one-dimensional direction of the plane.

In the invention according to the second aspect, the acoustic impedance is lowered while substantially maintaining an electro-mechanical coupling coefficient.

In a third aspect of the invention there is provided, in combination with the above first aspect, an ultrasonic probe wherein the piezoelectric elements in the composite piezoelectric element are arranged in a matrix shape in a two-dimensional direction within the plane.

In the invention according to the third aspect, the acoustic impedance is further lowered while substantially maintaining an electro-mechanical coupling coefficient.

In a fourth aspect of the invention there is provided, in combination with any of the above first to third aspects, an ultrasonic probe wherein the piezoelectric elements and the plate-like connector portion are formed by cutting a single piezoelectric material having the shape of a rectangular solid.

In the invention according to the fourth aspect, the piezoelectric elements and the plate-like connector portion are formed positively through a small number of manufacturing steps.

In a fifth aspect of the invention there is provided, in combination with any of the above first to fourth aspects, an ultrasonic probe wherein the piezoelectric elements are each PZT.

In the invention according to the above fifth aspect, the electro-mechanical coupling coefficient is made high.

In a sixth aspect of the invention there is provided, in combination with the above sixth aspect, an ultrasonic probe wherein the filler is resin.

In a seventh aspect of the invention there is provided, in combination with any of the above first to sixth aspects, an ultrasonic probe wherein the adhesive layer contains an epoxy resin adhesive.

In the invention according to the above seventh aspect, the composite piezoelectric elements and the complete reflector layer are bonded together strongly.

In an eighth aspect of the invention there is provided, in combination with any of the above first to seventh aspects, an ultrasonic probe wherein the composite piezoelectric element has two cut holes in positions near both ends in the array direction of the composite piezoelectric element plate surface, the two cut holes being formed in a thickness direction orthogonal to the array direction within the plane.

In the invention according to the above eighth aspect, an electrode portion formed on the composite piezoelectric element plate surface is separated into two electrode portions isolated from each other.

In a ninth aspect of the invention there is provided, in combination with the above eighth aspect, an ultrasonic probe wherein the adhesive layer and the complete reflector layer are cut in the irradiation direction at the positions of the cut holes in the array direction.

In the invention according to the above ninth aspect, the adhesive layer and the complete reflector layer are also allowed to function as two insulated electrode portions.

In a tenth aspect of the invention there is provided, in combination with the above ninth aspect, an ultrasonic probe wherein the cut holes are formed by cutting the composite piezoelectric element and the complete reflector layer from the complete reflector layer side, the composite piezoelectric clement and the complete reflector layer being joined together through the adhesive layer.

In the invention according to the above tenth aspect, the cut holes are formed by a simple manufacturing process.

In an eleventh aspect of the invention there is provided, in combination with any of the above first to tenth aspects, an ultrasonic probe wherein the complete reflector layer contains tungsten.

In the invention according to the above eleventh aspect, the complete reflector layer is rendered high in acoustic impedance and conductive.

In a twelfth aspect of the invention there is provided, combination with the above eleventh aspect, an ultrasonic probe further including a flexible printed circuit board at a plate surface position of the complete reflector layer opposed to the plate surface of the complete reflector layer where the adhesive layer is positioned.

In the invention according to the above twelfth aspect, the electrodes are taken out without exerting any influence on the adhesive layer.

In a thirteenth aspect of the invention there is provided, in combination with the above twelfth aspect, an ultrasonic probe wherein the flexible printed circuit board has an electric insulating layer disposed at each of the portions of the complete reflector layer which are in contact with the cut holes, a conductor foil disposed at the portion of the complete reflector layer which is in contact with the area sandwiched in between the two cut holes, and a conductor foil disposed at each of the portions of the complete reflector layer which are in contact with the areas expanding toward the ends from the two cut holes positioned in the array direction.

In the invention according to the above thirteenth aspect, both electrodes of earth terminal and drive terminal are taken out from only one surface of the complete reflector layer where the flexible printed circuit board is present.

In a fourteenth aspect of the invention there is provided, in combination with any of the above first to thirteenth aspects, an ultrasonic probe wherein the composite piezoelectric element has an acoustic matching layer on the other composite piezoelectric element plate surface in the irradiation direction.

In the invention according to the above fourteenth aspect, sensitivity is improved by the acoustic matching layer.

In a fifteenth aspect of the invention there is provided, in combination with the above fourteenth aspect, an ultrasonic probe wherein the acoustic matching layer has a rubber lens on an acoustic matching layer plate surface located on the side opposite to the composite piezoelectric element in the irradiation direction.

In the invention according to the above fifteenth aspect, a sound field converged in the thickness direction is created by the rubber lens.

In a sixteenth aspect of the invention there is provided an ultrasonic imaging apparatus including an ultrasonic probe for transmission of an ultrasonic echo to a subject and reception of the ultrasonic echo reflected from the subject, an image acquiring section for acquiring tomographic image information on the subject, a display section for displaying the tomographic image information, and a control section for controlling the transmission, the reception, the acquisition and the display, the ultrasonic probe including a composite piezoelectric element including piezoelectric elements arranged through gaps on a plane and a filler filled in each of the gaps, an adhesive layer positioned in an ultrasonic wave irradiation direction orthogonal to the plane, the adhesive layer being in contact with one plate surface of the composite piezoelectric element, and a complete reflector layer joined to the composite piezoelectric element through the adhesive layer to reflect an ultrasonic elastic oscillation generated in the composite piezoelectric element substantially toward the composite piezoelectric element plate surface, wherein the composite piezoelectric element has a plate-like connector portion constituted by only the piezoelectric elements, the plate-like connector portion covering the whole of the composite piezoelectric element.

According to the invention, since the plate-like connector portion constituted by only the piezoelectric elements in the composite piezoeleetric element is specularly polished to make concaves and convexes small, the adhesive layer sandwiched in between the plate-like connector portion and the complete reflector layer is made thin and uniform in thickness and hence it is possible to let the composite piezoelectric element generate an elastic oscillation of a quarter-wave resonance in a stable and positive manner and render the ultrasonic probe high in sensitivity and wide in frequency band.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the entire configuration of an ultrasonic imaging apparatus.

FIG. 2 is an appearance diagram showing the appearance of an ultrasonic probe.

FIG. 3 is an appearance diagram showing the appearance of only a functional element portion of the ultrasonic probe.

FIG. 4 is a sectional view showing a section of the functional element portion of the ultrasonic probe.

FIG. 5 is an appearance diagram showing the appearance of a composite piezoelectric element in the functional element portion.

FIGS. 6(A) and 6(B) are explanatory diagrams showing an elastic oscillation in the composite piezoelectric element portion and a complete reflector layer.

FIG. 7 is an explanatory diagram showing a relation between the thickness of an adhesive layer present between the composite piezoelectric element and the complete reflector layer and a frequency bandwidth.

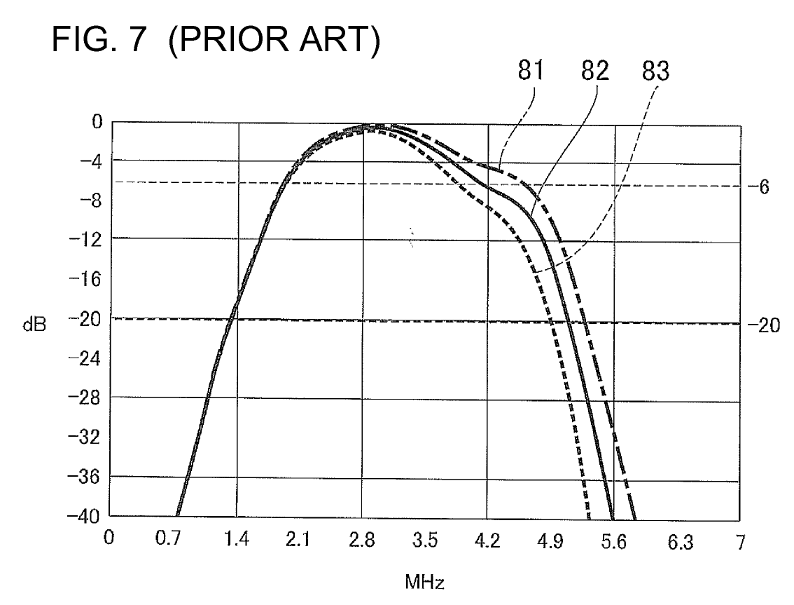

### DETAILED DESCRIPTION OF THE INVENTION

With reference to the accompanying drawings, various embodiments of an ultrasonic imaging apparatus according to the invention will be described below. The invention should not be limited to the following embodiments.

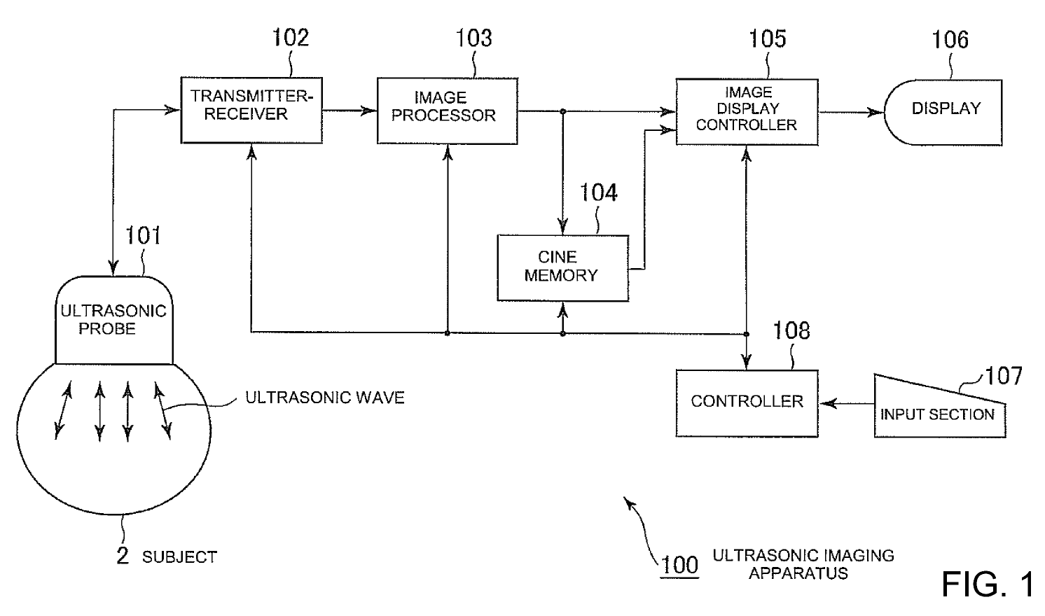

A description will be given about the entire configuration of an ultrasonic imaging apparatus 100 according to an embodiment of the invention. FIG. 1 is a block diagram showing the entire configuration of the ultrasonic imaging apparatus 100. The ultrasonic imaging apparatus 100 includes an ultrasonic probe 101, a transmitter-receiver section 102, an image processing section 103, a cine memory section 104, an image display control section 105, a display section 106, an input section 107, and a control section 108.

The ultrasonic probe 101 is a sector type probe for transmission and reception of an ultrasonic wave. It transmits an ultrasonic wave to an imaging section of the subject 2 and receives, as time-series sound rays, an ultrasonic echo reflected from the interior of the subject 2. Further, the ultrasonic probe 101 repeats scanning while successively switching transmitting and receiving directions that spreads in a fan shape centered on the probe to acquire two-dimensional tomographic image information of the imaging section.

The transmitter-receiver section 102 is connected to the ultrasonic probe 101 through a coaxial cable and produces an electrical signal for driving the composite piezoelectric element in the ultrasonic probe 101. This electrical signal has a transmitting waveform in the generation of an ultrasonic wave and there is used a burst waveform including plural pulses with a repeating frequency approximate to the resonance frequency of the composite piezoelectric element. Further, the transmitter-receiver section 102 performs a first-stage amplification of the received ultrasonic echo signal.

The image processing section 103 produces a B mode image in real time from the ultrasonic echo signal amplified in the transmitter-receiver section 102. As concrete processing contents there are a delay addition processing for the received ultrasonic echo signal, an A/D (analog/digital) conversion processing, and a process of writing digital information after the conversion, as B mode image information, into the cine memory section 104 which will be described later. When color flow mapping as one of doppler processings is to be performed, the image processing section 103 extracts phase shift information on the ultrasonic echo signal and calculates in real time flow information associated with various points of the imaging section such as speed, power and variance.

The cine memory section 104 is an image memory for storing the B mode image information produced in the image processing section and flow information.

The image display control section 105 performs display frame rate conversion for the B mode image information produced in the image processing section 103 and flow information and controls the shape and position of an image displayed on the display section 106.

The display section 106 is constituted by a CRT (Cathode Ray Tube) or LCD (Liquid Crystal Display) to display for an operator the image information after the display frame rate conversion and control of the image display shape and position performed by the image display control section 105.

The input section 107 transmits to the control section 108 an operation input signal indicative of operation performed by the operator, e.g., an operation input signal for selecting whether scanning in B mode is to be performed or scanning of color flow mapping is to be performed.

In accordance with the operation input signal transmitted from the input section 107 and pre-stored program and data the control section 108 controls the operation of each of the components of the ultrasonic imaging apparatus described above.

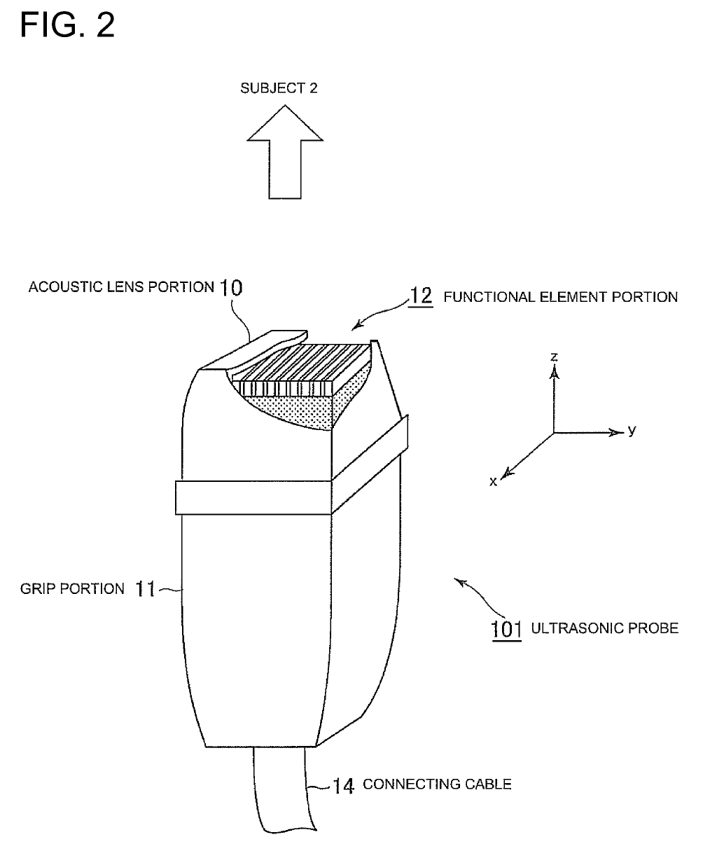

FIG. 2 is an appearance diagram showing the appearance of the sector type ultrasonic probe 101. The ultrasonic probe 101 includes an acoustic lens portion 10, a grip portion 11, a functional element portion 12 and a connecting cable 14. In the acoustic lens portion 10, for ease of understanding, the functional element portion 12 as a built-in portion is partially exposed unlike the actual state. The xyz axis coordinates illustrated in the drawing are common in coordinate axis to xyz axis coordinates illustrated in the subsequent drawing, illustrating a positional relation among the drawings.

The acoustic lens portion 10 is disposed on the side where the ultrasonic probe 101 comes into contact with the subject 2 and causes an ultrasonic wave generated in the functional element portion 12 to be directed to the subject 2 efficiently. The acoustic lens portion 10 is made of a material which has an acoustic impedance substantially intermediate between the subject 2 and the functional element portion 12. The acoustic lens portion 10 has a convex lens shape at its portion that comes into contact with the subject 2 to converge the ultrasonic wave incident on the subject 2.

The grip portion 11 is gripped by the operator and makes the acoustic lens portion come closely into contact with the subject 2. In the interior of the grip portion 11 is disposed, for example, a flexible printed circuit board for connection of electrodes of the functional element portion 12 and coaxial cables included in the connecting cable 14.

The connecting cable 14 is a bundle of plural coaxial cables and provides an electric connection between the transmitter-receiver section 102 and the functional element portion 12.

The functional clement portion 12 generates an ultrasonic wave and applies it to the subject 2. At the same time, it receives an ultrasonic echo reflected from the interior of the subject 2.

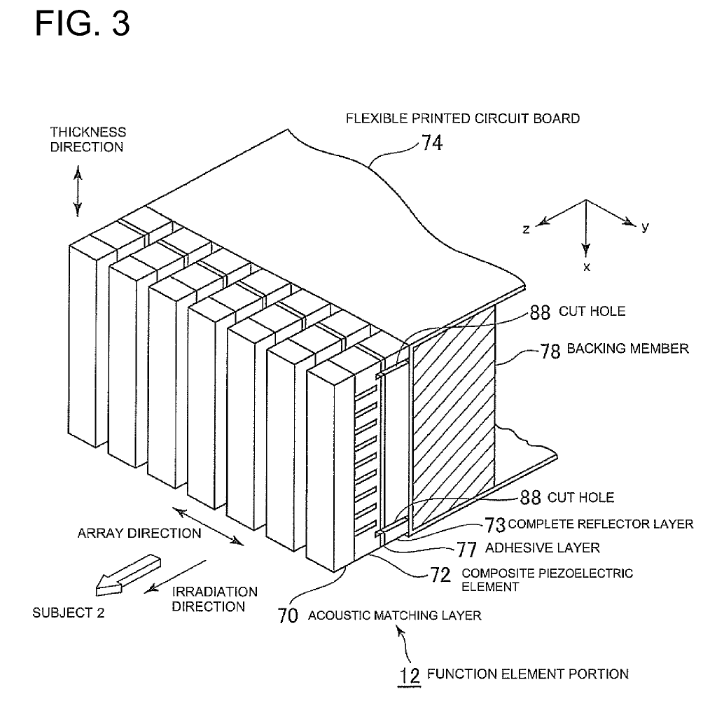

FIG. 3 is an appearance diagram showing the appearance of only the functional element portion 12. The functional element portion 12 includes an acoustic matching layer 70, a composite piezoelectric element 72, an adhesive layer 77, a complete reflector layer 73, a backing member 78 and a flexible printed circuit board 74. The acoustic matching layer 70, the composite piezoelectric element 72 and the complete reflector layer 73 each having the shape of a rectangular solid are stacked in z-axis direction which is an ultrasonic wave irradiating direction, providing a stacked structure. Like acoustic matching layers 70, composite piezoelectric elements 72 and complete reflector layers 73, having such a stacked structure, are arranged in the array direction, namely, in y-axis direction.

In each composite piezoelectric element 72, which has the shape of a rectangular solid, piezoelectric elements such as PZT (piezoelectric zirconate titanate) are arranged one-dimensionally in x-axis direction that is the thickness direction of the rectangular solid. Further, epoxy resin is filled in gaps of the piezoelectric elements arranged in the thickness direction. The composite piezoelectric element 72 functions as a piezoelectric element, including the thus-filled epoxy resin. The composite piezoelectric element 72 is approximately equal in electromechanical coupling coefficient to PZT and is smaller in acoustic impedance than PZT. Consequently, the difference in acoustic impedance from for example the acoustic matching layer 70, which is a load on the composite piezoelectric element 72, becomes small and the resonance frequency characteristic has a wide frequency band.

The acoustic matching layer 70 is joined to a plate surface of the composite piezoelectric element 72 in z-axis direction that is the irradiation direction where the subject 2 is present. The acoustic matching layer 70 has an acoustic impedance substantially intermediate between the composite piezoelectric element 72 and the acoustic lens portion 10 shown in FIG. 2. The acoustic matching layer 70 has a thickness corresponding approximately to a quarter wavelength of the ultrasonic wave passing therethrough to suppress reflection at a boundary surface different in acoustic impedance. Although in FIG. 3 the acoustic matching layer is shown as a single layer, two or multiple such layers may also be adopted.

The adhesive layer 77 is for bonding the composite piezoelectric element 72 and the complete reflector layer 73 with each other and it is an epoxy resin adhesive for example.

The complete reflector layer 73 is for completely reflecting an elastic oscillation generated in the composite piezoeleetric element 72 and it is bonded to the plate surface of the composite piezoelectric element 72 in the direction opposite to the irradiation direction. The complete reflector layer 73 completely reflects in the direction of irradiating the subject 2 the ultrasonic wave generated in the composite piezoelectric element 72 and radiated in the direction opposite to the direction of the subject 2, thereby increasing the ultrasonic power incident on the subject 2. In view of the purpose of reflecting the ultrasonic wave completely it is preferable that the complete reflector layer 73 be formed of a material high in acoustic impedance, e.g. tungsten. Cut holes 88 are present in positions close to both ends in the thickness direction of the composite piezoelectric element 72, adhesive layer 77 and complete reflector layer 73. For example, the cut holes 88 are formed by bonding the composite piezoelectric element 72 and the complete reflector layer 73 with each other through the adhesive layer 77 and by subsequent cutting from the complete reflector layer 73 side with use of a diamond wheel for example. As to the function of the cut holes 88, it will be described in detail later.

The backing member 78 holds the complete reflector layer 73 and the flexible printed circuit board 74.

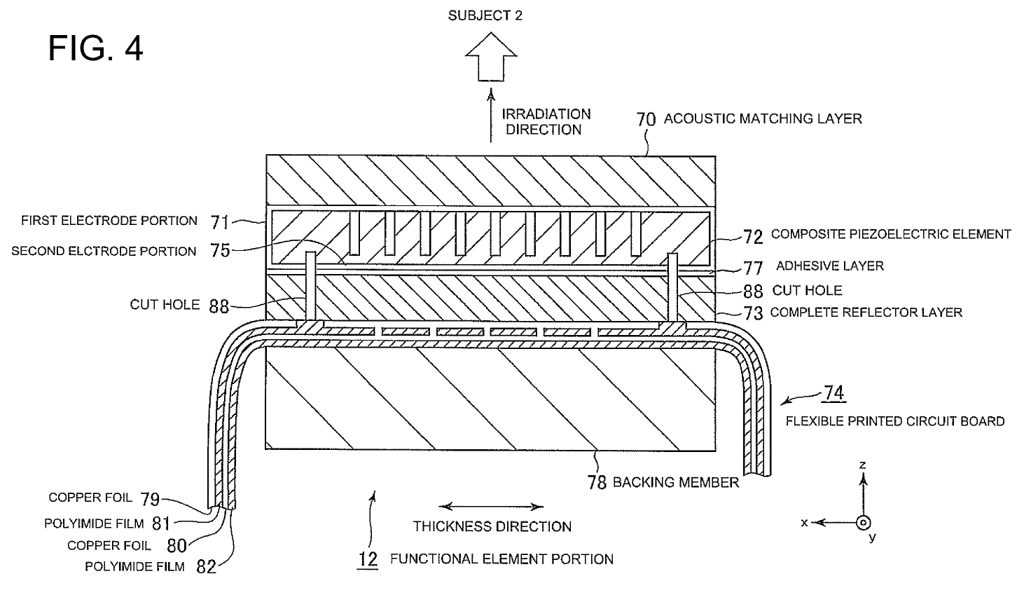

The flexible printed circuit board 74 is a four-layer flexible printed circuit board disposed between the complete reflector layer 73 and the backing member 78. The flexible printed circuit board 74 is drawn out oppositely to the subject 2, i.e., backward, along a side face in the thickness direction of the backing member 78 and is connected with the connecting cable 14. In FIG. 3 there is shown an earthing surface of the flexible printed circuit board 74 that is covered with copper foil uniformly along a side face in the thickness direction of the backing member 78. On the back side of the earthing surface of the flexible printed circuit board which side is not shown there are disposed plural copper foil patterns extending in z-axis direction in parallel, the copper foil patterns serving as electrodes which are independent for each composite piezoelectric element 72 in the array direction. The details of this point will be described below with reference to FIG. 4.

FIG. 4 is a sectional view showing the details of xz section of the functional element portion 12. In FIG. 4, as compared with FIG. 3, a first electrode portion 71 and a second electrode portion 75 are shown in more detail in the composite piezoelectric element 72, and copper foils 79, 80 and polyimide films 81, 82 are shown in more detail in the flexible printed circuit board 74.

The first electrode portion 71 and the second electrode portion 75 are disposed so as to surround the xz section of the composite piezoelectric element 72 and both are electrically insulated from each other by the cut holes 88. The first electrode portion 71 is disposed on the plate surface in the irradiation direction of the composite piezoelectric element 72, the side face in x-axis direction and the plate surface extending toward the x-axis end from the two cut holes 88 present on the complete reflector layer 73 side, while the second electrode portion 75 is disposed between the two cut holes 88 formed in the complete reflector layer 73-side plate surface of the composite piezoelectric element 72. The first and second electrode portions 71, 75 are formed, for example, by sputtering.

The thickness in z-axis direction, i.e., in the irradiation direction, of the adhesive layer 77 is set at a value of the order of several μm. This thickness is almost equal to the size of concaves and convexes formed on the plate surface which is in contact with the first and second electrode portions 71, 75 and concaves and convexes formed on the plate surface which is in contact with the complete reflector layer 73 to be described later. Thus, although the adhesive layer 77 is a insulator containing an epoxy resin adhesive, the first and second electrode portions 71, 75 and the complete reflector layer 73 partially contact each other the concave/convex portion of the plate surface, having electrical conductivity in z-axis direction.

The complete reflector layer 73 contains tungsten having a high hardness. The complete reflector layer 73 is specularly polished on its plate surface located on the side where the composite piezoelectric element 72 is present. Therefore, the concaves and convexes of the plate surface of the complete reflector layer 73 on the side where the composite piezoelectric element 72 is present are suppressed to several μm or so.

Since the tungsten that constitutes the complete reflector layer 73 possesses high electrical conductivity, it also functions to electrically connect the copper foil of the flexible printed circuit board 74 to be described later and the composite piezoelectric element 72 with each other. The cut holes 88 are formed in x-axis positions of the adhesive layer 77 and the complete reflector layer 73, providing an electrically insulated state between the central portion in x-axis direction and ends in the same direction of the adhesive layer 77 and the complete reflector layer 73.

The flexible printed circuit board 74 is disposed on the backing member 78 side of the complete reflector layer 73 and applies voltage to the composite piezoelectric element 72. For example, the flexible printed circuit board 74 is made up of four layers that are the copper foils 79, 80 and the polyimide films 81, 82. The copper foils 79 and 80 are insulated from each other by the polyimide film 81. The copper foil 79 is positioned at x-axis ends of the complete reflector layer 73 from the cut holes 88. At the x-axis ends of the complete reflector layer 73 the copper foil 80 is positioned on the back side of the copper foil 79 through the polyimide film 81, while at the x-axis central portion of the complete reflector layer 73 the copper foil 80 is present also on the same surface as the copper foil 79 through a through hole having electrical conductivity.

The copper foil 79 is electrically connected to the first electrode portion 71 through the complete reflector layer 73 having electrical conductivity and the adhesive layer 77, while the copper foil 80 is electrically connected to the second electrode portion 75 through the complete reflector layer 73 having electrical conductivity and the adhesive layer 77.

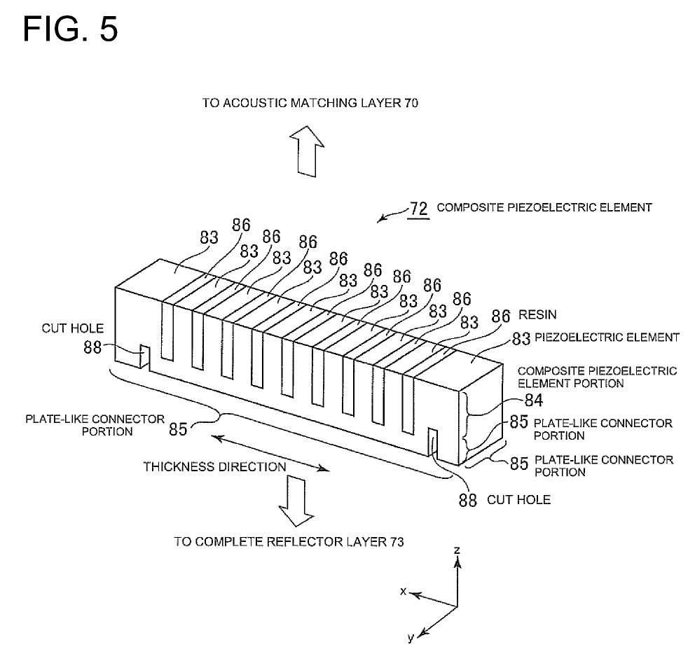

FIG. 5 is an explanatory diagram showing the appearance only the composite piezoelectric element 72. The first and second electrode portions 71, 75 are not shown. The composite piezoelectric element 72 includes a composite piezoelectric element portion 84 and a plate-like connector portion 85 in z-axis direction that is an ultrasonic wave irradiating direction.

The composite piezoelectric element portion 84 is positioned on the acoustic matching layer 70 side of the composite piezoelectric element 72 and has a structure such that piezoelectric elements 83 of PZT for example and resin 86 are arranged alternately in x-axis direction.

The plate-like connector portion portion 85 is positioned on the complete reflector layer 73 side of the composite piezoelectric element 72 and has a plate-like structure wherein an xy-axis plane is constituted by only the piezoelectric element portion 83. For example, the plate-like connector portion 85 is formed by cutting from the acoustic matching layer 70 side a piezoelectric material of the same rectangular solid shape as the composite piezoelectric element 72 shown in FIG. 5. In this case, the cutting depth in the irradiation direction is set at approximately 80% of the length in the irradiation direction of the composite piezoelectric element 72.

The complete reflector layer 73 side of the composite piezoelectric element 72 is specularly polished using an abrasive. This plate surface is constituted in the plate-like connector portion 85 by only the piezoelectric elements 83 having uniform hardness. Therefore, concaves and convexes on the complete reflector layer 73-side plate surface of the composite piezoeleetric element layer 72 can be made small by specular polishing.

In the case where the plate-like connector portion 85 is not present, two materials widely different in hardness, namely, the piezoelectric elements 83 formed of a ceramic material and the resin 86, are coexistent on the complete reflector layer 73-side plate surface of the composite piezoelectric element 72. Such a plate surface is difficult to be specularly polished uniformly and it is not easy to form small concaves and convexes of the order of several μm.

The complete reflector layer 73-side plate surface of the composite piezoelectric element 72 and the composite piezoelectric element 72-side plate surface of the complete reflector layer 73 are each formed of a single material and therefore concaves and convexes can be made small by uniform specular polishing. As a result, the adhesive layer 77 formed between the composite piezoelectric element 72 and the complete reflector layer 73 is uniform in thickness and is thin.

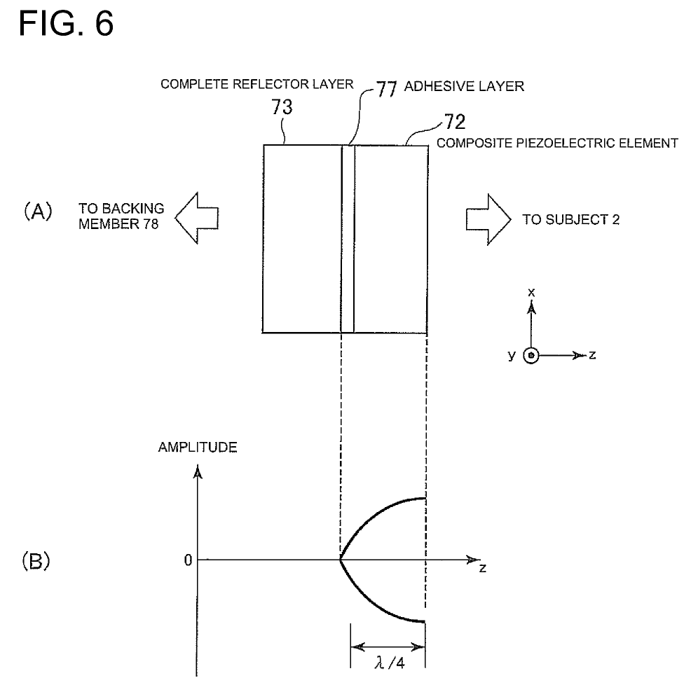

The operation of the functional element portion 12 will now be outlined with reference to FIGS. 6(A) and 6(B). FIGS. 6(A) and 6(B) are explanatory diagrams showing a partial structure and an elastic oscillation of the functional element portion 12. FIG. 6(A) shows xz-axis sections of the composite piezoelectric element 72, adhesive layer 77 and complete reflector layer 73. The thickness in z-axis direction of the composite piezoelectric element 72 is determined by the center frequency of the generated ultrasonic wave and is approximately 100 μm. On the other hand, as to the thickness of the adhesive layer 77, since the opposed plate surfaces of the composite piezoelectric element 72 and the complete reflector layer 73 are both specularly polished uniformly, the thickness and variations in thickness of the adhesive layer are maintained within several μm or so.

Since the complete reflector layer 73 is formed of a material high in acoustic impedance as compared with the composite piezoelectric element 72, the ultrasonic wave incident from the composite piezoelectric element 72 is almost reflected. Consequently, there exists no ultrasonic wave generated in the composite piezoelectric element 72.

FIG. 6(B) shows an amplitude distribution of an elastic oscillation generated in z-axis direction in both composite piezoelectric element 72 and complete reflector layer 73. In FIG. 6(B), a z-axis position is plotted along the abscissa axis and the magnitude of the amplitude of the elastic oscillation is plotted along the ordinate axis. The z-axis coordinate position plotted along the abscissa axis corresponds to the z-axis position of the composite piezoelectric element 72 and the complete reflector layer 73 shown in FIG. 6(A).

With voltage applied between the first and second electrode portions 71, 75, the composite piezoelectric element 72 excites a resonant oscillation. This resonant oscillation forms a standing wave with the subject 2 side being substantially a free oscillation side and the backing member 78 side being substantially a fixed end because the subject 2 side is a low acoustic impedance of the acoustic matching layer 70 and the backing member 78 side is a high acoustic impedance side of the complete reflector layer 73.

FIG. 6(B) illustrates a standing wave whose amplitude becomes maximum at the subject 2-side end of the composite piezoelectric element 72 and becomes zero on the backing member 78 side of the composite piezoelectric clement 72. From the same figure it is seen that in the composite piezoelectric element 72 there occurs a standing wave with the z-axis thickness of the composite piezoelectric element 72 being a quarter wavelength (λ/4) in a resonant state. In the complete reflector layer 73 the amplitude becomes zero because there is no incidence of an elastic oscillation.

All the energy of the elastic oscillation generated in the composite piezoelectric element 72 is released to the subject 2 side, so that it is possible to improve the sensitivity of the composite piezoelectric element 72. Besides, since the adhesive layer 77 is thin and uniform, the generated ultrasonic wave is large in amplitude without impairing the function of the adhesive layer 77 as a fixed end of the elastic oscillation of the complete reflector layer 73.

According to this embodiment, as described above, the plate-like connector portion 85 constituted by only the piezoelectric elements 83 is provided on the plate surface of the composite piezoelectric element 72 opposed to the complete reflector layer 73 and the plate surface of the plate-like connector portion 85 opposed to the complete reflector layer 73, together with the opposed plate surface of the complete reflector layer 73, is specularly polished to make concaves and convexes small, so that it is possible to make the thickness of the adhesive layer 77 small and uniform and hence possible to make the ultrasonic probe high in sensitivity and wide in frequency band.

In the composite piezoelectric element 72 according to this embodiment, the piezoelectric elements are arranged one-dimensionally in the thickness direction, which array is called 2-2 type. However, there may be adopted a modification wherein piezoelectric elements are arranged in matrix form within the xy-axis plane and resin is filled in gap portions between adjacent piezoelectric elements, whereby it is possible to obtain a composite piezoelectric element of a low acoustic impedance.

Many widely different embodiments of the invention may be configured without departing from the spirit and the scope of the present invention. It should be understood that the present invention is not limited to the specific embodiments described in the specification, except as defined in the appended claims.

1. An ultrasonic probe comprising;
a composite piezoelectric element comprising a plurality of piezoelectric elements arranged through gaps on a plane and a filler filled in each of the gaps;
an adhesive layer positioned in an ultrasonic wave irradiating direction orthogonal to the plane, said adhesive layer being in contact with a first plate surface of said composite piezoelectric element; and
a complete reflector layer joined to said composite piezoelectric element through said adhesive layer to reflect an ultrasonic elastic oscillation generated in said composite piezoelectric element substantially toward said first composite piezoelectric element plate surface, wherein said composite piezoelectric element comprises a plate-like connector portion comprising said plurality of piezoelectric elements, said plate-like connector portion covering said composite piezoelectric element.

2. An ultrasonic probe according to claim 1, wherein said plurality of piezoelectric elements are arranged in a one-dimensional direction of the plane.

3. An ultrasonic probe according to claim 1, wherein said plurality of piezoelectric elements are arranged in a matrix shape in a two-dimensional direction within the plane.

4. An ultrasonic probe according to claim 1, wherein said plurality of piezoelectric elements and said plate-like connector portion are formed by cutting a single piezoelectric material having a shape of a rectangular solid.

5. An ultrasonic probe according to claim 1 wherein said plurality of piezoelectric elements are each PZT.

6. An ultrasonic probe according to claim 1, wherein said filler is resin.

7. An ultrasonic probe according to claim 1, wherein said adhesive layer contains an epoxy resin adhesive.

8. An ultrasonic probe according to claim 1, wherein said composite piezoelectric element comprises two cut holes in positions near both ends in an array direction of said first composite piezoelectric element plate surface, said two cut holes being formed in a thickness direction orthogonal to the array direction within the plane.

9. An ultrasonic probe according to claim 2, wherein said composite piezoelectric element comprises two cut holes in positions near both ends in an array direction of said first composite piezoelectric element plate surface, said two cut holes being formed in a thickness direction orthogonal to the array direction within the plane.

10. An ultrasonic probe according to claim 3, wherein said composite piezoelectric element comprises two cut holes in positions near both ends in an array direction of said first composite piezoelectric element plate surface, said two cut holes being formed in a thickness direction orthogonal to the array direction within the plane.

11. An ultrasonic probe according to claim 4, wherein said composite piezoelectric element comprises two cut holes in positions near both ends in an array direction of said first composite piezoelectric element plate surface, said two cut holes being formed in a thickness direction orthogonal to the array direction within the plane.

12. An ultrasonic probe according to claim 5, wherein said composite piezoelectric element comprises two cut holes in positions near both ends in an array direction of said first composite piezoelectric element plate surface, said two cut holes being formed in a thickness direction orthogonal to the array direction within the plane.

13. An ultrasonic probe according to claim 8, wherein said adhesive layer and said complete reflector layer are cut in the irradiation direction at the positions of said cut holes in the array direction.

14. An ultrasonic probe according to claim 13, wherein said cut holes are formed by cutting said composite piezoelectric element and said complete reflector layer from a complete reflector layer side, said composite piezoelectric element and said complete reflector layer being joined together through said adhesive layer.

15. An ultrasonic probe according to claim 1, wherein said complete reflector layer contains tungsten.

16. An ultrasonic probe according to claim 15, further comprising a flexible printed circuit board at a plate surface position of said complete reflector layer opposed to said first plate surface of said complete reflector layer where said adhesive layer is positioned.

17. An ultrasonic probe according to claim 16, wherein said flexible printed circuit board comprises an electric insulating layer disposed at each portion of said complete reflector layer which is in contact with said cut holes, a conductor foil disposed at a portion of said complete reflector layer which is in contact with an area sandwiched in located between said two cut holes, and a conductor foil disposed at each portion of said complete reflector layer which is in contact with an area expanding toward ends from said two cut holes positioned in the array direction.

18. A piezoelectric probe according to claim 1, wherein said composite piezoelectric element comprises an acoustic matching layer on a second composite piezoelectric element plate surface in the irradiation direction.

19. An ultrasonic probe according to claim 18, wherein said acoustic matching layer comprises a rubber lens on an acoustic matching layer plate surface located on a side opposite said composite piezoelectric element in the irradiation direction.

20. An ultrasonic imaging apparatus comprising:
an ultrasonic probe configured to transmit an ultrasonic echo to a subject and to receive the ultrasonic echo reflected from the subject;
an image acquiring section configured to acquire tomographic image information on the subject;
a display section configured to display the tomographic image information; and
a control section configured to control the transmission, the reception, the acquisition and the display, wherein said ultrasonic probe comprises:
a composite piezoelectric element including a plurality of piezoelectric elements arranged through gaps on a plane and a filler filled in each of the gaps;
an adhesive layer positioned in an ultrasonic wave irradiating direction orthogonal to the plane, said adhesive layer being in contact with a plate surface of said plurality of composite piezoelectric element; and
a complete reflector layer joined to said composite piezoelectric element through said adhesive layer to reflect an ultrasonic elastic oscillation generated in said composite piezoelectric element substantially toward said composite piezoelectric element plate surface, wherein said composite piezoelectric element comprises a plate-like connector portion comprising said plurality of piezoelectric elements, said plate-like connector portion covering said composite piezoelectric element.
